(19) United States
(12) Patent Application Publication
(10) Pub. No.: US 2024/0156503 A1
(43) Pub. Date: May 16, 2024

(54) SURGICAL IMPACTING TOOL COUPLINGS

(71) Applicant: DePuy Synthes Products, Inc., Raynham, MA (US)

(72) Inventors: Eric Schmitt, Palm Beach Gardens, FL (US); Cory Shulaw, Warsaw, IN (US); David Narducci, Jupiter, FL (US); Jon M. Edwards, Stuart, FL (US); Nathanael Conner, Roanoke, IN (US)

(21) Appl. No.: 18/487,326

(22) Filed: Oct. 16, 2023

Related U.S. Application Data
(60) Provisional application No. 63/425,911, filed on Nov. 16, 2022.

(51) Int. Cl. A61B 17/92

(52) U.S. Cl. CPC A61B 17/92; A61B 2017/00486

(57) ABSTRACT

Various exemplary surgical impacting tool couplings and methods of using surgical impacting tool couplings are provided. In general, an adapter can be configured to be releasably attached to a surgical impacting tool handpiece. The adapter can be configured to couple to a surgical implement configured to impact bone. The surgical impacting tool handpiece, such as a handpiece of an orthopedic impactor, is configured to drive impacting of the surgical implement relative to bone.

### CROSS REFERENCE TO RELATED APPLICATIONS

The present application claims priority to U.S. Provisional Patent Application No. 63/425,911 entitled “Surgical Impacting Tool Couplings” filed on Nov. 16, 2022, which is hereby incorporated by reference in its entirety.

### FIELD

The present disclosure relates generally to surgical impacting tool couplings.

### BACKGROUND

In the field of orthopedics, prosthetic devices, such as artificial joints, are often implanted or seated in a patient's bone cavity. The cavity is typically formed during surgery before a prosthetic device is seated or implanted by, for example, a physician or other medical professional removing and/or compacting existing bone to form the cavity. The prosthetic device, which can also be referred to as a prosthesis, usually includes a stem or other protrusion that is inserted into the cavity.

To create the cavity, a physician or other medical professional may use a broach, chisel, or other surgical implement conforming to the shape of the stem of the prosthetic device. In general, the surgical implement is impelled into the implant area to form the cavity. One technique for impelling the surgical implement includes a physician or other medical professional manual hammering the surgical impacting tool to impel the surgical implement into the implant area. Another technique for creating the prosthetic cavity relies on computer-controlled robotic arms for creating the cavity instead of using manual power provided by a physician or other medical professional. Another technique for creating the prosthetic cavity is to drive the surgical implement pneumatically, e.g., by compressed air. Another technique for creating the prosthetic cavity relies on a linear compressor to compress air on a single stroke basis and then, after a sufficient pressure is created, to release the air through a valve and onto a striker to impel the surgical implement.

The broach, chisel, or other surgical implement can be removably coupled to the surgical impacting tool to, for example, allow for surgical implements of different sizes and/or shapes to be used with the surgical impacting tool in different surgical procedures to help accommodate a particular patient's needs, to allow for replacement of surgical implements that become worn, damaged, or otherwise undesirable for future use without having to replace a remainder of the surgical impacting tool, and/or to accommodate a surgeon's personal preference of surgical implements. However, various techniques for creating the prosthetic cavity that impel the surgical implement, such as the four techniques discussed above, can loosen the surgical implement's removable coupling to the surgical impacting tool due to the force required to impel the surgical implement. Such loosening may cause the surgical implement to unexpectedly become decoupled from the surgical impacting tool during a surgical procedure, may cause the surgical implement to shake or otherwise move in unintentional direction(s) and thus cause patient harm and/or adversely affect cavity formation, and/or may hinder cavity formation by not allowing the surgical implement to receive and be impelled at full intended force.

Accordingly, there remains a need for improved surgical impacting tools.

### SUMMARY

In general, surgical impacting tool couplings and methods of using surgical impacting tool couplings are provided.

In one aspect, a surgical device is provided that in one embodiment includes an adapter configured to have a portion thereof releasably seated in a cavity formed in a handpiece of a surgical impacting tool configured to drive impacting of bone via the adapter. The adapter includes a proximal portion having a substantially cylindrical shape, a substantially circular cross-sectional shape, and a first diameter. The adapter also includes a distal portion having at least four sides defining an outer perimeter of the distal portion, and the distal portion has a second diameter that is greater than the first diameter. The adapter also includes a neck located between the proximal portion and the distal portion. The neck has a substantially cylindrical shape, a substantially circular cross-sectional shape, and a third diameter that is less than the first diameter and less than the second diameter. The surgical device can have any number of variations.

In another embodiment, a surgical device includes a handpiece of a surgical impacting tool and includes an adapter configured to have a portion thereof releasably seated in a cavity formed in the handpiece. The handpiece is configured to drive impacting of bone via the adapter. The adapter includes a proximal portion having a substantially cylindrical shape, a substantially circular cross-sectional shape, and a first diameter. The adapter also includes a distal portion having at least four sides defining an outer perimeter of the distal portion, and the distal portion has a second diameter that is greater than the first diameter. The adapter also includes a neck located between the proximal portion and the distal portion. The neck has a substantially cylindrical shape, a substantially circular cross-sectional shape, and a third diameter that is less than the first diameter and less than the second diameter. The surgical device can have any number of variations.

In another embodiment, a surgical device includes an adapter. The adapter includes a proximal portion configured to be releasably seated in a cavity formed in a handpiece of a surgical impacting tool, and the adapter includes a reduced diameter portion as compared to a first diameter of a first portion of the adapter proximal to the reduced diameter portion and a second diameter of a second portion of the adapter distal to the reduced diameter portion. The reduced diameter portion has a proximal fillet and a distal fillet, and a radius of the distal fillet has a greater radius than the proximal fillet. The surgical device can have any number of variations.

In another embodiment, a surgical device includes an adapter and a handpiece of a surgical impacting tool. The adapter includes a proximal portion configured to be releasably seated in a cavity formed in the handpiece, and the adapter includes a reduced diameter portion as compared to a first diameter of a first portion of the adapter proximal to the reduced diameter portion and a second diameter of a second portion of the adapter distal to the reduced diameter portion. The reduced diameter portion has a proximal fillet and a distal fillet, and a radius of the distal fillet has a greater radius than the proximal fillet. The handpiece includes first and second pawls configured to move relative to a housing of the handpiece between a first position, in which the first and second pawls are not engaged with the reduced diameter portion of the adapter, and a second position, in which the first and second pawls are not engaged with the reduced diameter portion of the adapter. The adapter is in a locked position relative to the handpiece with the first and second pawls in the second position. The surgical device can have any number of variations.

In another aspect, a surgical method is provided that in one embodiment includes releasably attaching an adapter to a handpiece of a surgical impacting tool by moving the adapter into a cavity of the handpiece substantially along a longitudinal axis defined by the cavity, and driving impacting of a surgical implement relative to bone. The surgical implement is coupled to the adapter, and a direction of the impacting being substantially along the longitudinal axis defined by the cavity. The adapter is configured to have a portion thereof releasably seated in the cavity, which is formed in the handpiece. The handpiece is configured to drive impacting of bone via the adapter. The adapter includes a proximal portion having a substantially cylindrical shape, a substantially circular cross-sectional shape, and a first diameter. The adapter also includes a distal portion having at least four sides defining an outer perimeter of the distal portion, and the distal portion has a second diameter that is greater than the first diameter. The adapter also includes a neck located between the proximal portion and the distal portion. The neck has a substantially cylindrical shape, a substantially circular cross-sectional shape, and a third diameter that is less than the first diameter and less than the second diameter. The surgical method can have any number of variations.

In another embodiment, a surgical method includes releasably attaching an adapter of to a handpiece of a surgical impacting tool by moving the adapter into a cavity of the handpiece substantially along a longitudinal axis defined by the cavity, and driving impacting of a surgical implement relative to bone. The surgical implement is coupled to the adapter, and a direction of the impacting is substantially along the longitudinal axis defined by the cavity. The adapter includes a proximal portion configured to be releasably seated in a cavity formed in a handpiece of a surgical impacting tool, and the adapter includes a reduced diameter portion as compared to a first diameter of a first portion of the adapter proximal to the reduced diameter portion and a second diameter of a second portion of the adapter distal to the reduced diameter portion. The reduced diameter portion has a proximal fillet and a distal fillet, and a radius of the distal fillet has a greater radius than the proximal fillet. The surgical method can have any number of variations.

### BRIEF DESCRIPTION OF DRAWINGS

The present invention is described by way of reference to the accompanying figures which are as follows:

FIG. 1 is a side view of an embodiment of a surgical impacting tool handpiece;

FIG. 2 is a top view of the handpiece of FIG. 1;

FIG. 3 is a front view of the handpiece of FIG. 1;

FIG. 4 is a back view of another embodiment of a surgical impacting tool handpiece;

FIG. 5 is a front view of the handpiece of FIG. 4 with one embodiment of an adapter releasably attached thereto;

FIG. 6 is a side view of the handpiece and adapter of FIG. 5;

FIG. 7 is a perspective view of the handpiece and adapter of FIG. 5;

FIG. 8 is another perspective view of the handpiece and adapter of FIG. 5;

FIG. 9 is a perspective view of one embodiment of an adapter;

FIG. 10 is a cross-sectional view of the adapter of FIG. 9;

FIG. 11 is another cross-sectional view of the adapter of FIG. 9;

FIG. 12 is a side view of the adapter of FIG. 9;

FIG. 13 is a proximal end view of the adapter of FIG. 9;

FIG. 14 is a view of a portion of the adapter of FIG. 12;

FIG. 15 is a perspective view of another embodiment of an adapter;

FIG. 16 is a cross-sectional view of the adapter of FIG. 15;

FIG. 17 is a side view of the adapter of FIG. 15;

FIG. 18 is another cross-sectional view of the adapter of FIG. 15;

FIG. 19 is a proximal end view of the adapter of FIG. 15;

FIG. 20 is a perspective view of yet another embodiment of an adapter;

FIG. 21 is a cross-sectional view of the adapter of FIG. 20;

FIG. 22 is a side view of the adapter of FIG. 20;

FIG. 23 is another side view of the adapter of FIG. 20;

FIG. 24 is a cross-sectional view of the adapter of FIG. 22;

FIG. 25 is another perspective view of the adapter of FIG. 20;

FIG. 26 is a perspective view of another embodiment of an adapter;

FIG. 27 is a proximal end view of the adapter of FIG. 26;

FIG. 28 is a cross-sectional view of the adapter of FIG. 26;

FIG. 29 is a side view of the adapter of FIG. 26;

FIG. 30 is a perspective, partial view of the adapter of FIG. 26;

FIG. 31 is a perspective view of one embodiment of a surgical implement;

FIG. 32 is a perspective view of another embodiment of a surgical implement;

FIG. 33 is a perspective, cross-sectional view of one embodiment of a locking assembly releasably coupled with the adapter of FIG. 9;

FIG. 34 is another perspective, cross-sectional view of the locking assembly and the adapter of FIG. 33;

FIG. 35 is a perspective view, cross-sectional view of the locking assembly and the adapter of FIGS. 33 and 34 with a housing of the locking assembly rotated;

FIG. 36 is a cross-sectional view of the locking assembly and the adapter of FIGS. 33 and 34;

FIG. 37 is an exploded perspective view of another embodiment of a locking assembly;

FIG. 38 is a perspective view of a housing of the locking assembly of FIG. 37;

FIG. 39 is a side, cross-sectional view of the housing of FIG. 38;

FIG. 40 is a perspective view of a base of the locking assembly of FIG. 37;

FIG. 41 is a side, cross-sectional view of the base of FIG. 40;

FIG. 42 is a perspective view of a biasing element of the locking assembly of FIG. 32;

FIG. 43 is a side view of the biasing element of FIG. 42;

FIG. 44 is a perspective view of a support of the locking assembly of FIG. 37;

FIG. 45 is a side view of the support of FIG. 44;

FIG. 46 is a perspective view of a pawl of the locking assembly of FIG. 37; and

FIG. 47 is a side, cross-sectional view of the pawl of FIG. 46.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a dimension may not be a precise value but nevertheless be considered to be at about that value (for example, may be within +/−0.5% of the value) due to any number of factors such as manufacturing tolerances and sensitivity of measurement equipment. Sizes and shapes of the systems and devices, and the components thereof, can depend at least on the size and shape of components with which the systems and devices will be used.

Various exemplary surgical impacting tool couplings and methods of using surgical impacting tool couplings are provided. In general, an adapter can be configured to be releasably attached to a surgical impacting tool handpiece. The adapter being releasably attachable to the surgical impacting tool handpiece allows the surgical impacting tool handpiece to be releasably attachable to a variety of different adapters. Each of the adapters can be different from one another in one or more aspects such as length, configured to be have a surgical implement releasably attached thereto or having a surgical implement non-removably attached thereto, etc., thereby allowing for a particular adapter to be selected by a surgeon (or other medical professional) for optimal desired impacting in a particular surgical procedure being performed on a particular patient's bone.

The adapter can be configured to releasably couple to a surgical implement configured to impact bone. The surgical impacting tool handpiece, such as a handpiece of an orthopedic impactor, is configured to drive impacting of the surgical implement relative to bone. The surgical impacting tool handpiece releasably attached to the adapter is configured to provide a force to the surgical implement, via the adapter, to drive the impacting of the surgical implement. The surgical implement configured to be attached to the adapter can be a broach, chisel, or other surgical implement. Further, each of the surgical implements configured to be attached to an adapter can be different from one another in one or more aspects, such as shape, size, etc., thereby allowing for a particular surgical implement to be selected by a surgeon (or other medical professional) for optimal desired impacting in a particular surgical procedure being performed on a particular patient's bone.

In some embodiments, instead of the surgical implement being releasably attached to the adapter, the surgical implement can be non-releasably attached to the adapter to allow the surgical impacting tool handpiece to be used with a variety of different surgical implements by being attachable to a variety of different adapters.

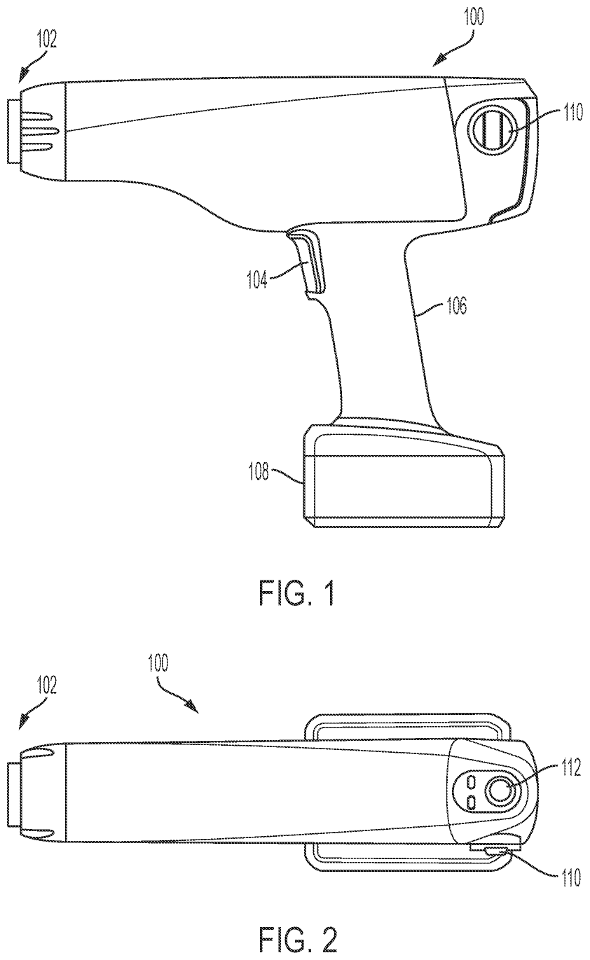

FIGS. 1-3 illustrate one embodiment of a surgical impacting tool handpiece 100 including a locking assembly configured to releasably attach to an adapter. The handpiece of a surgical impacting tool is a handpiece of an orthopedic impactor in this illustrated embodiment, but as mentioned above, the surgical impacting tool can be another type of surgical impacting tool. The locking assembly is located at a forward or distal end 102 of the surgical impacting tool handpiece 100. Embodiments of the locking assembly are discussed further below.

The surgical impacting tool handpiece 100 includes an actuator 104 configured to be actuated to drive a surgical implement attached to an adapter that is releasably attached to the handpiece 100 via the locking assembly. The actuator 104 in this illustrated embodiment includes a trigger on a handle 106 of the handpiece 100, but other surgical impacting tools can be actuated in other ways. In an exemplary embodiment, the handpiece 100 is configured to provide forward impacting, in which a forward force is provided by the handpiece 100 for impacting in a forward direction, and rearward impacting, in which a rearward force is provided by the 1 handpiece 100 for impacting in a rearward direction. The forward and rearward impacting can be cyclical with sequential repeated forward and rearward impacts. In some embodiments, the handpiece 100 can be configured to provide only one of forward impacting and rearward impacting.

A power source 108 is configured to releasably attached to the handle 106 of the handpiece 100. The power source 108 includes a battery in this illustrated embodiment, but other power sources are possible. In other embodiments, the handpiece 100 can be releasably attachable to a power source in another way, such as by being plugged into a power source. In still other embodiments, the power source can be non-releasably attached to the handpiece 100, such as by a battery being non-removably disposed in, e.g., the handle 106.

The handpiece 100 includes an energy selector 110 on the handle 106 of the handpiece 100. The energy selector 110 includes a rotary dial in this illustrated embodiment but can have other configurations, such as a lever, a button, etc. The energy selector 110 is configured to allow an energy level to be selected, e.g., high energy or low energy.

The handpiece 100 includes a frequency control 112 on the handle 106 of the handpiece 100. The frequency control 112 includes a button in this illustrated embodiment but can have other configurations, such as a lever, a rotary dial, etc. The frequency control 112 is configured to allow a frequency of impacts to be selected by a user, e.g., slow impacts or fast impacts.

The handpiece 100 can have additional or alternate features. Various exemplary embodiments of surgical impacting tool handpieces including additional or alternate features are further described, for example, in U.S. patent application Ser. No. 17/319,700 entitled “Surgical Impacting Tool Interfaces” filed May 13, 2021, U.S. Pat. Pub. No. 2013/0161050 entitled “Electric Motor Driven Tool For Orthopedic Impacting” published Jun. 27, 2013, U.S. Pat. No. 10,912,597 entitled “Orthopedic Adapter For An Electric Impacting Tool” issued Feb. 9, 2021, U.S. Pat. No. 11,083,512 entitled “Orthopedic Impacting Device Delivering A Controlled, Repeatable Impact” issued Aug. 10, 2021, U.S. Pat. Pub. No. 11,134,962 entitled “Orthopedic Impacting Device Having A Launched Mass Delivering A Controlled, Repeatable & Reversible Impacting Force” issued Oct. 5, 2021, U.S. Pat. No. 8,393,409 entitled “Electric Motor Driven Tool For Orthopedic Impacting” issued Mar. 12, 2013, U.S. Pat. No. 8,936,105 entitled “Electric Motor Driven Tool For Orthopedic Impacting” issued Jan. 20, 2015, and U.S. Pat. No. 8,695,726 entitled “Electric Motor Driven Tool For Orthopedic Impacting” issued Apr. 15, 2014, which are hereby each incorporated by reference in their entirety.

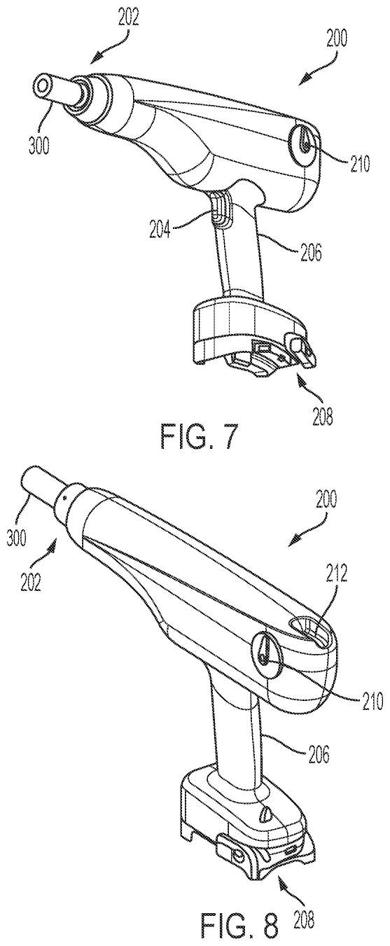

FIGS. 4-8 illustrate another embodiment of a surgical impacting tool handpiece 200 including a locking assembly configured to releasably attach to an adapter 300. FIGS. 4-8 show the adapter 300 releasably attached to the handpiece 200 via the locking assembly. The handpiece 200 of the surgical impacting tool is a handpiece of an orthopedic impactor in this illustrated embodiment, but as mentioned above, the surgical impacting tool can be another type of surgical impacting tool. The handpiece 200 is generally configured and used similar to the handpiece 100 of FIG. 1, e.g., includes the locking assembly at a forward or distal end 202 of the handpiece 200 and includes an actuator 204, a handle 206, an energy selector 210 (which is a lever in this illustrated embodiment), and a frequency control 212 (which is a dial in this illustrated embodiment). The handpiece 200 in this illustrated embodiment is configured at a bottom end 208 of the handpiece 200 to releasably attach to a power source, similar to the handpiece 100 being releasably attachable to the power source 108 as discussed above. Embodiments of the locking assembly are discussed further below.

The adapter 300 in this illustrated embodiment is configured to releasably attach to a surgical implement to impact bone. In other embodiments, the adapter 300 can be non-releasably attached to a surgical implement.

The adapter 300, as well as other embodiments of adapters described herein, can be formed from a rigid biocompatible material, such as stainless steel, titanium, or other material. The rigid biocompatible material may allow the adapter to be repeatedly impacted without deforming or breaking over the course of its use with one or more surgical impacting tool handpieces in the performance of one or more surgical procedures.

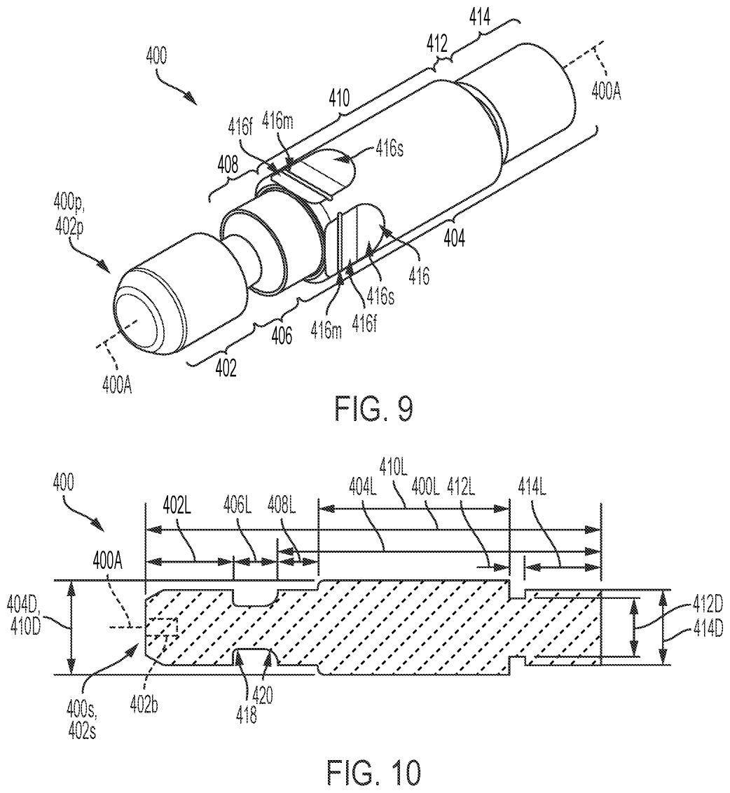

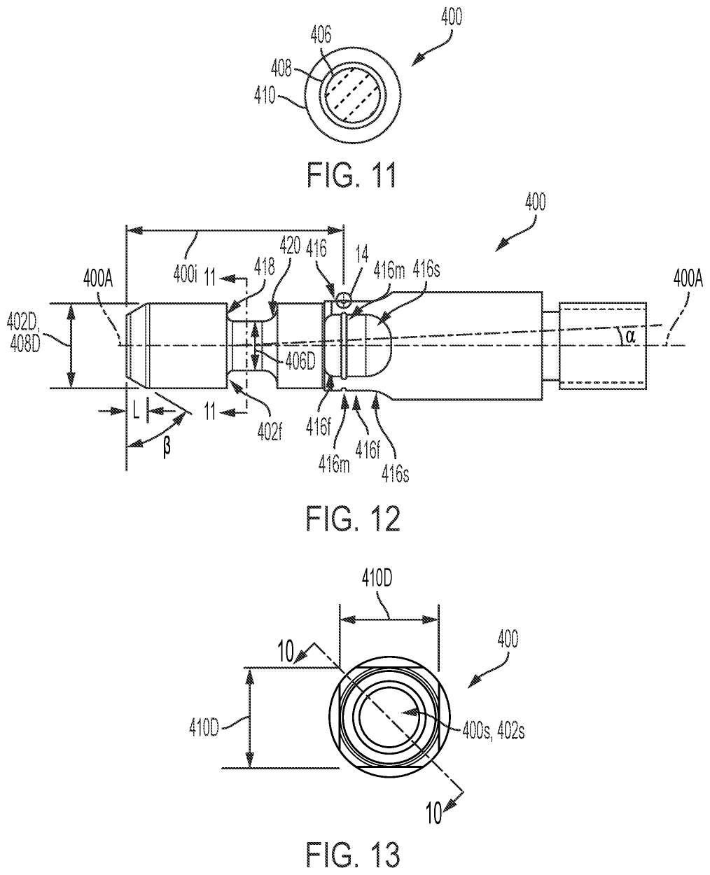

FIGS. 9-13 illustrate another embodiment of an adapter 400 configured to be releasably attached to a surgical impacting tool handpiece, such as the surgical impacting tool handpiece 100 of FIGS. 1-3, the surgical impacting tool handpiece 200 of FIGS. 4-8, or another surgical impacting tool handpiece. The adapter 400 includes a proximal portion 402, a distal portion 404, and an intermediate portion 406 located between the proximal and distal portions 402, 404. As shown in FIGS. 9, 10, and 12, the intermediate portion 406 is immediately distal to the proximal portion 402, and the distal portion 404 is immediately distal to the intermediate portion 406.

The proximal, distal, and intermediate portions 402, 404, 406 are aligned along a longitudinal axis 400A of the adapter 400. This longitudinal alignment may facilitate longitudinally-directed impacting, whether the impacting is in a distal or forward direction or in a proximal or rearward direction.

The proximal portion 402 of the adapter 400 has a substantially cylindrical shape and has a substantially circular cross-sectional shape. A person skilled in the art will appreciate that a shape may not be precise, e.g., not precisely cylindrical or precisely circular (for example, be within +/−0.5% of the cylinder and circular diameter), but nevertheless be considered to be substantially that shape due to any number of factors, such as manufacturing tolerances and sensitivity of measurement equipment. As discussed further below, the substantially cylindrical shape and substantially circular cross-sectional shape of the proximal portion 402 may reduce angular play of the adapter 400 with respect to a surgical impacting tool handpiece within which the proximal portion 402 is disposed. An angular play at an angle a shown in FIG. 12 would not result with use of the adapter 400 but is illustrated to demonstrate angular play. Less angular play between an adapter and a surgical impacting tool handpiece within which the adapter is partially disposed may allow for a more secure fit between the adapter and handpiece and/or may facilitate impacting more precisely along the adapter's longitudinal axis, and thus provide for more predictably directed impacting on bone, since the adapter's angular movement relative to the longitudinal axis is reduced.

The proximal portion 402 has a diameter 402D and a longitudinal length 402L. The proximal portion 402 in this illustrated embodiment is chamfered at its proximal end 402p. A proximal end 400p of the adapter 400, which is defined by the proximal portion 402, is thus chamfered. The proximal portion 402 therefore has a smaller diameter at the proximal end 402p thereof than in a remainder of the proximal portion 402 that is located distal to the chamfering. The diameter 402D of the proximal portion 402 (labeled in FIG. 12) thus defines a maximum diameter of the proximal portion 402. The adapter 400 having a chamfered portion at its proximal end 400p may facilitate insertion of the adapter 400 into a cavity formed in a surgical impacting tool handpiece since, as discussed further below, the adapter 400 can be inserted into the cavity in a proximal direction. Additionally, as also discussed further below, relative sizes of the cavity and the adapter 400 are such that the maximum diameter of an insertion portion 400i of the adapter 400 that is inserted into the cavity is just less than a diameter of the cavity at least at a distal opening thereof. The chamfered portion of the adapter 400 may thus make the adapter 402 easier to initially insert into the cavity since the chamfered portion reduces the adapter's diameter at its proximal end 400p. The insertion portion 400i of the adapter 400 includes the proximal portion 402, the intermediate portion 406, and a proximal portion of the distal portion 404, as discussed further below.

As shown in FIG. 12, the chamfered portion of the adapter 400 in the proximal portion 402 is at an angle β. The angle β is about 60° in this illustrated embodiment but can be another angle. A person skilled in the art will appreciate that values may not be precisely at a value but nevertheless be considered to be about that value (for example, be within +/−0.5% of the value) due to any number of factors, such as manufacturing tolerances and sensitivity of measurement equipment.

As also shown in FIG. 10, the chamfered portion has a longitudinal length L. The longitudinal length L of the chamfered portion is about one-fifth (⅕) the longitudinal length 402L of the proximal portion 402 in this illustrated embodiment, but the lengths L, 402L can have another ratio.

In other embodiments, the proximal end 400p of the adapter 400, and thus the proximal end 402p of the proximal portion 402, is not chamfered and the proximal portion 402 has a substantially equal diameter along its entire longitudinal length 402L. A person skilled in the art will appreciate that values may not be precisely equal but nevertheless be considered to be substantially equal (for example, be within +/−0.5% of each other) due to any number of factors, such as manufacturing tolerances and sensitivity of measurement equipment.

A proximal-facing surface 402s of the proximal portion 402 at its proximal end 402p defines a proximal-facing surface 400p of the adapter 400 at its proximal end 400p. With the adapter 400 releasably attached to a surgical impacting tool handpiece, the proximal-facing surface 402s of the proximal portion 402, and thus the proximal-facing surface 400s of the adapter 400, is configured to be impacted to provide a forward or distally directed impact force, as discussed further below.

The proximal-facing surface 402s of the proximal portion 402, and thus the proximal-facing surface 400p of the adapter 400, is a continuous solid surface in this illustrated embodiment, as shown in FIGS. 9 and 13. The continuous solid surface may facilitate impacting of the proximal-facing surface 402s since an entire area within a perimeter of the proximal-facing surface 402s is available for impacting.

In other embodiments, the proximal-facing surface 402s of the proximal portion 402 has one or more blind bores formed therein, such as a single blind bore 402b (shown in phantom in FIG. 10) in a center of the proximal-facing surface 402s, a plurality of blind bores in a circular pattern centered around a center of the proximal-facing surface 402s, or other position. The one or more blind bores are configured to seat therein one or more corresponding protrusions of a surgical impacting tool handpiece in which the adapter 400 is partially disposed. The seating of the one or more protrusions within the one or more blind bores may help further reduce adapter play and/or may help ensure that the adapter 400 has been inserted into the handpiece at a proper orientation relative to the handpiece since the adapter 400 may be prevented from being disposed as much as possible within the handpiece unless the one or more bores seat the one or more protrusions therein, e.g., because the protrusion(s) will otherwise abut the proximal-facing surface 402s. Each of the one or more blind bores, and thus each of the corresponding one or more protrusions, can have any of a variety of shapes, e.g., a substantially cylindrical shape, a substantially cube shape, a substantially triangular pyramid shape, a substantially conical shape, etc.

With the adapter 400 releasably attached to a surgical impacting tool handpiece, a distal-facing surface 402f of the proximal portion 402 is configured to be impacted to provide a rearward or proximally directed impact force, as discussed further below.

The distal portion 404 of the adapter 400 includes a first portion 408 that is distal to the intermediate portion 406, a second portion 410 that is distal to the first portion 408, a third portion 412 that is distal to the second portion 410, and a fourth portion 414 that is distal to the third portion 412. The longitudinal length 404L of the distal portion 404 equals a sum of longitudinal lengths 408L, 410L, 412L, 414L of the first, second, third, and fourth portions 408, 410, 412, 414. A longitudinal length 400L of the adapter 400 equals a sum of the longitudinal lengths 402L, 404L, 406L of the proximal, distal, and intermediate portions 402, 404, 406.

The longitudinal length 408L of the third portion 408 is substantially equal to the longitudinal length 406L of the intermediate portion 406. The longitudinal length 402L of the proximal portion 402 is thus about twice the length 408L of the third portion 408.

A diameter 408D of the first portion 408 of the distal portion 404 is substantially equal to the diameter 402D of the proximal portion 402, as shown in FIG. 12. A diameter of the adapter 400 immediately distal to the intermediate portion 406 and immediately proximal to the intermediate portion 406 is thus substantially equal. The intermediate portion 406 therefore has a diameter 406D less than the diameters 402D, 408D of its immediately proximal and distal portions 402, 408 and thus defines a reduced diameter portion. The substantially equal diameters of the proximal and first portions 408, 402 may reduce angular play of the adapter 400 with respect to a surgical impacting tool handpiece within which the proximal portion 402, intermediate portion 406, and first portion 408 are disposed. The intermediate portion 406 having a smaller diameter 406D than the proximal portion 402 and the first portion 408 of the distal portion 404 facilitates locking of the adapter 400 within a surgical impacting tool handpiece, as discussed further below. The diameter 406D of the intermediate portion 406 can be, for example, in a range of about 55% to about 60% (e.g., about 57% or another percentage) of the diameter 402D of the proximal portion and in a range of about 40% to about 50% (e.g., about 45% or another percentage) of the second diameter.

A diameter 410D of the second portion 410 of the distal portion 404 defines a maximum diameter of the distal portion 404 and also a maximum diameter of the adapter 400, as shown in FIG. 10. The diameter 410D of the second portion 410 is greater than the diameter 408L of the first portion 408 of the distal portion 404 and thus also greater than the diameter 406D of the intermediate portion 406 and the diameter 402D of the proximal portion 402. The maximum diameter of the adapter 400 proximal to the second portion 410, e.g., the diameter 402D, 408D of the proximal portion 402 and the first portion 408, is therefore less than the diameter 410D of the second portion 404. The adapter 400 having a smaller diameter proximal to the second portion 410 may facilitate insertion of the adapter 400 within a surgical impacting tool handpiece by abutting against a surface of the handpiece, as discussed further below. In this illustrated embodiment the diameter 410D of the second portion 410 is about 20% greater than the maximum diameter of the adapter 400 proximal to the second portion 410, but another ratio of these diameters is possible, e.g., in a range of about 15% to about 20%, about 16%, etc.

A diameter 414D of the fourth portion 414 of the distal portion 404 is less than the diameter 410D of the second portion 410, and a diameter 412D of the third portion 412 of the distal portion 404 is less than the diameter 414D of the fourth portion 414. The third portion 412 therefore has a diameter 412D less than the diameters 410D, 414D of its immediately proximal and distal portions 410, 414 and thus defines a reduced diameter portion of the distal portion 404.

Each of the first, third, and fourth portions 408, 412, 414 of the adapter's distal portion 404 has a substantially cylindrical shape and has a substantially circular cross-sectional shape. As discussed further below, the substantially cylindrical shape and substantially circular cross-sectional shape of the first portion 408 may reduce angular play of the adapter 400 with respect to a surgical impacting tool handpiece within which the first portion 408 is disposed. The substantially cylindrical shape and substantially circular cross-sectional shape of the third and fourth portions 412, 414 may reduce angular play of the adapter 400 with respect to a surgical implement to which the adapter 400 is releasably coupled.

The distal portion 404 includes a portion having four sides 416. The second portion 410 of the adapter's distal portion 404 includes a first, proximal portion having the four sides 416 (one of the sides 416 is obscured in the figures). The second portion 410 of the adapter's distal portion 404 also includes a second, distal portion that is distal to the second portion's first, proximal portion. The second portion's second, distal portion has a substantially cylindrical shape and has a substantially circular cross-sectional shape.

Each of the four sides 416 is substantially planar in at least a proximal portion thereof. The planar surfaces 416f of the sides 416 are each substantially parallel to the adapter's longitudinal axis 400A. In contrast to the four sides 416, the adapter 400 proximal to the distal portion 404, e.g., the proximal and intermediate portions 402, 406 of the adapter, is substantially cylindrical and thus has a continuous circumferential surface, e.g., a continuous circumferential surface around the proximal portion 402 and a continuous circumferential surface around the intermediate portion 406.

The planar surfaces 416f of the sides 416 are configured to be at least partially disposed within a surgical impacting tool handpiece, as discussed further below. The planar surfaces 416f can thus help prevent rotation of the adapter 400 relative to the handpiece with the insertion portion 400i of the adapter 400 fully and properly disposed within the handpiece.

Each of the four sides 416 in this illustrated embodiment also includes a sloped portion 416s in a distal portion thereof. The sloped portion 416s slopes radially outwardly toward the maximum diameter 404D, 410D of the distal portion.

The four sides 416 are spaced equidistantly around a perimeter of the distal portion 404, around a perimeter of the second portion 410. The four sides 416 in this illustrated embodiment are each identical to one another and define a substantially square cross-sectional shape with the planar surfaces. In other embodiments, the four sides 416 can define a rectangular cross-sectional shape. In such embodiments, two of the sides 416 can be the same as one another and different from the other two sides 404s, e.g., different in size since two sides 416 are longer than the other two sides 416 due to the rectangular cross-sectional shape.

In other embodiments, the second portion 410 of the adapter's distal portion 404 does not include a substantially cylindrical portion and instead only has four sides 416 that are substantially planar.

As mentioned above, an insertion portion 400i of the adapter 400 is configured to be disposed within a surgical impacting tool handpiece. The adapter 400 includes a marker configured to indicate a level of the adapter's insertion into the handpiece. A user may therefore be able to visually confirm, by visually observing the marker, whether the adapter 400 is in a locked position relative to the handpiece at a location as proximally as far as possible within the handpiece so as to be fully and properly disposed therein. With the insertion portion 400i of the adapter 400 in the locked position relative to the handpiece, the marker is configured to align with a feature of the handpiece, e.g., a distal-most surface of the handpiece, a surface surrounding a cavity in which the adapter 400 is being disposed, etc., and thereby indicate that the adapter 400 is in the locked position.

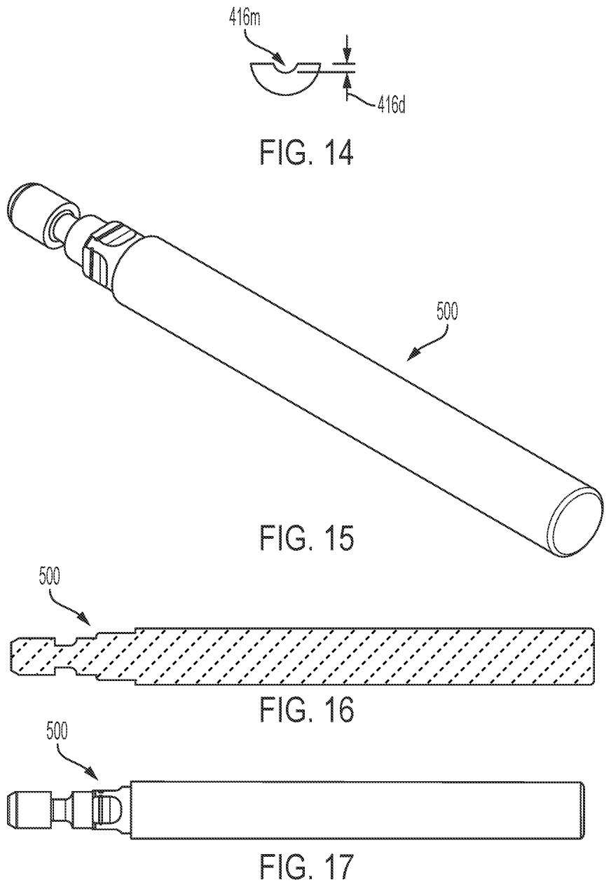

The marker can have a variety of configurations. In this illustrated embodiment, the marker 416m includes a groove formed in each of the four sides 416 of the distal portion 404 and, more particularly, in each the planar surface 416f. The markers 416m (except for one obscured marker) are illustrated in FIGS. 9, 12, and 14. The markers 416m each have a depth 416d. The markers 416m are each a straight groove extending substantially perpendicular to the adapter's longitudinal axis 400A. The markers 416m thus extend substantially perpendicular to a direction in which the adapter 400 is moved into the handpiece (and/or in which the handpiece is advanced over the adapter 400). This substantially perpendicular position of the markers 416m can allow the markers 416m to be closer to the handpiece the more of the insertion portion 400i is within the handpiece. Thus, when the markers 416m align with the feature of the handpiece, the markers 416m have moved as close as possible to the handpiece and thus indicate that the insertion portion 400i has been fully and properly disposed within the handpiece.

In other embodiments, the one or more markers can include a marking printed on the side 416, a marking etched on the side 416, and a sticker on the side 416. In other embodiments, the adapter 400 in a portion immediately proximal to the one or more markers, e.g., at least a proximal portion of the planar surfaces 416f, is a first color, the adapter 400 in a portion immediately distal to the one or more markers, e.g., at least a distal portion of the planar surfaces 416f, is a second, different color, and a junction of the first and second colors defines the one or more markers. The first color becoming no longer visible indicates that the insertion portion 400i has been fully and properly disposed within the handpiece.

The adapter 400 is configured to be seated in a surgical impacting tool handpiece at one or more predetermined angular orientations relative to the handpiece. A surgical implement coupled to the adapter 400, and thus operatively coupled to the surgical impacting tool handpiece via the adapter 400, can therefore be attached to the surgical impacting tool handpiece at a plurality of predetermined angular orientations relative to the surgical impacting tool handpiece. Depending on one or more factors such as surgeon preference, which hand (left or right) of a user is holding the surgical impacting tool, which bone of a patient the surgical implement will be impacting, and a position of a patient relative to a user of the surgical impacting tool, a certain angular orientation of the surgical implement may be more desirable than another angular orientation of the surgical implement.

A number of the sides 416, and in particular the planar surfaces 416f thereof, defines a number of the predetermined angular orientations. The adapter 400 in the illustrated embodiment is thus configured to be seated in a surgical impacting tool handpiece at four predetermined angular orientations relative to the handpiece: about zero degrees, about ninety degrees, about one hundred eighty degrees, and about two hundred seventy degrees. Thus, in this illustrated embodiment, each of the predetermined angular orientations is about ninety degrees apart from one another.

The distal portion 404 of the adapter 400 is configured to releasably couple to a surgical implement. The adapter 400 can thus be coupled to any of a variety of surgical implements, thereby increasing versatility of the adapter 400. A person skilled in the art will appreciate that a surgical implement can releasably couple to the adapter 400, and other embodiments of adapters described herein, in a variety of ways. For example, a surgical implement can be configured to seat in the third portion 412 to releasably couple the surgical implement to the adapter 400. The third portion 412 of the adapter 400 having a smaller diameter 412D than the second and fourth portions 410, 414 facilitates locking of the adapter 400 with respect to a surgical implement releasably coupled to the adapter 400 since the larger diameters 410D, 414D can urge the surgical implement to remain seated in the reduced diameter portion defined by the third portion 412 until removal of the surgical implement from the adapter 400 is desired.

In some embodiments, the distal portion 404 of the adapter 400 can have a thread, e.g., a thread in the fourth portion 414, configure to threadably mate with a thread of a surgical implement to releasably couple to the surgical implement. In this illustrated embodiment the distal portion 404 is unthreaded.

In other embodiments, instead of being configured to releasably couple to a surgical implement, the adapter 400 can have a distally-extending surgical implement non-releasably coupled thereto. The surgical implement being non-releasably coupled to the adapter 400 may help prevent the surgical implement from becoming dislodged from or shifted in position with respect to the adapter 400 during impacting.

The intermediate portion (also referred to herein as a “neck”) 406 of the adapter 400 has a substantially cylindrical shape, a substantially circular cross-sectional shape, a diameter 406D, and a longitudinal length 406L. As discussed further below, the substantially cylindrical shape and substantially circular cross-sectional shape of the intermediate portion 406 may reduce angular play of the adapter 400 with respect to a surgical impacting tool handpiece within which the intermediate portion 406 is disposed.

As shown in FIG. 10, the longitudinal length 406L of the intermediate portion 406 is less than the longitudinal length 402L of the proximal portion 402. The longitudinal length 402L of the proximal portion 402 is about twice the longitudinal length 406L of the intermediate portion 406 in this illustrated embodiment, but the lengths 402L, 406L can have another ratio.

As shown in FIG. 12, the diameter 406D of the intermediate portion 406 is less than the diameter 402D of the proximal portion 402. The intermediate portion 406 having a smaller diameter than the proximal portion 402 (and a distal-most portion of the distal portion 404) facilitates locking of the adapter 400 within a surgical impacting tool handpiece, as discussed further below.

As shown in FIGS. 10 and 12, the intermediate portion 406 has a distal-facing concave fillet 418 at its proximal end and a proximal-facing concave fillet 420 at its distal end 420. The fillets 418, 420 are configured to help distribute stress over a broader area and effectively make the adapter 400 more durable and capable of bearing larger loads without breaking.

Due to the fillets 418, 420, the intermediate portion 406 has a larger diameter at each of its proximal and distal ends than in a reminder of the intermediate portion 406 located between its proximal and distal ends. The diameter 406D of the intermediate portion 406 (labeled in FIG. 12) thus defines a minimum diameter of the intermediate portion 406. The intermediate portion's diameter is still less than the diameter 402D of the proximal portion 402 and less than a diameter of a distal-most portion (e.g., first portion 408) of the distal portion 404 along the entire length 406L of the intermediate portion 406, as also shown in FIGS. 10 and 12.

The proximal-facing fillet 420 slopes at a radius that is greater than a radius of the sloped distal-facing fillet 418. The smaller radius at the sloped distal-facing fillet 418 may maximize an amount of distal-facing surface 402f of the proximal portion 402 available for impacting thereon to provide a rearward or proximally directed impact force, as discussed further below. The larger radius at the proximal-facing fillet 420 may increase a strength of the intermediate portion 406, and thus help prevent breakage of the adapter 400 at the reduced diameter portion 406. In this illustrated embodiment, the radius of the proximal-facing fillet 420 is about 65% greater than the radius of the distal-facing fillet 418. With the adapter 400 releasably coupled to a surgical impacting tool handpiece and used in impacting, the adapter's stress concentration may be greatest at the intermediate portion's proximal and distal ends. Further, the stress concentration may be greater at the intermediate portion's distal end than at the intermediate portion's proximal end. The fillets 418, 420 are thus located at the intermediate portion's proximal and distal ends to help distribute stress and thereby provide for a more durable adapter 400.

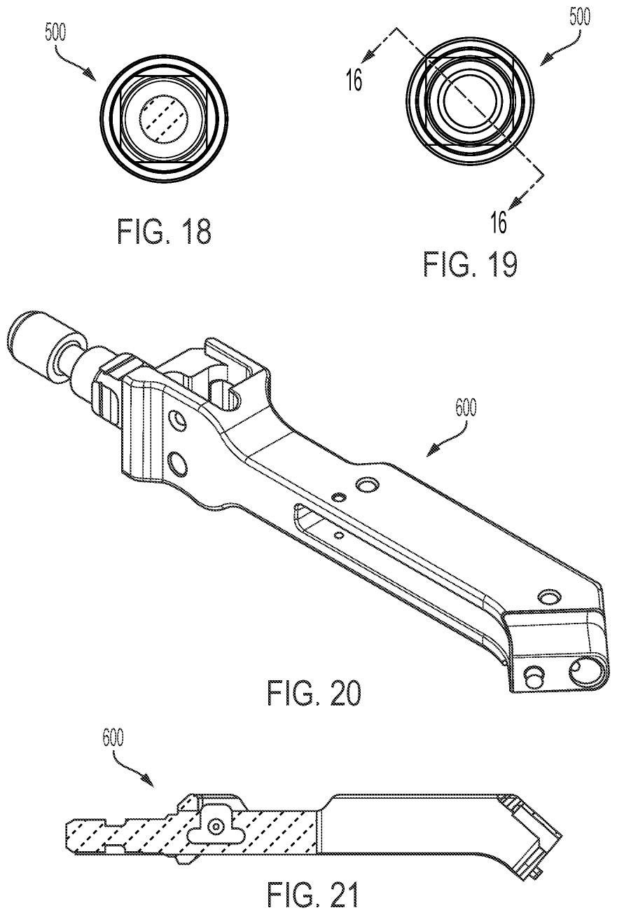

FIGS. 15-19 illustrate another embodiment of an adapter 500 configured to be releasably attached to a surgical impacting tool handpiece, such as the surgical impacting tool handpiece 100 of FIGS. 1-3, the surgical impacting tool handpiece 200 of FIGS. 4-8, or another surgical impacting tool handpiece. The adapter 500 of FIGS. 15-19 is configured and used similar to that discussed above with respect to the adapter 400 of FIGS. 9-13, e.g., includes a proximal portion, a distal portion, and an intermediate portion located between the proximal and distal portions. However, the adapter 500 differs from the adapter 400 of FIGS. 9-13 in that the distal portion of the adapter 500 has a longer longitudinal length than the distal portion 404 of the adapter 400, and the adapter 500 omits the third and fourth portions 412, 414 of the adapter 400.

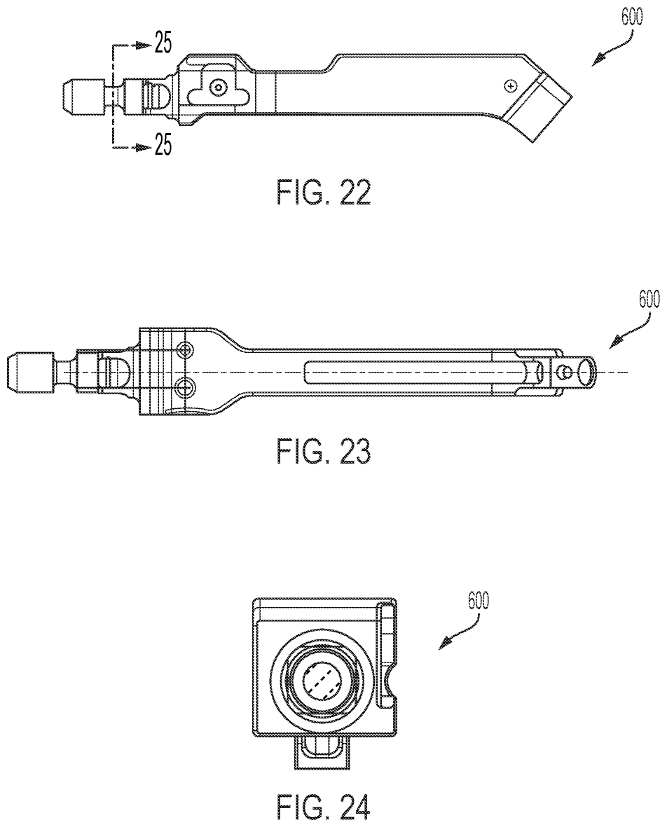

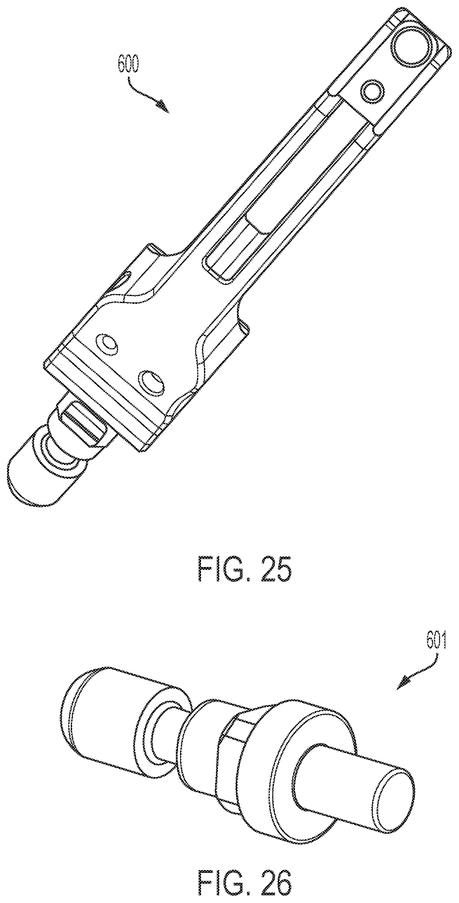

FIGS. 20-25 illustrate another embodiment of an adapter 600 configured to be releasably attached to a surgical impacting tool handpiece, such as the surgical impacting tool handpiece 100 of FIGS. 1-3, the surgical impacting tool handpiece 200 of FIGS. 4-8, or another surgical impacting tool handpiece. The adapter 600 of FIGS. 20-25 is configured and used similar to that discussed above with respect to the adapter 400 of FIGS. 9-13, e.g., includes a proximal portion, a distal portion, and an intermediate portion located between the proximal and distal portions. However, the adapter 600 differs from the adapter 400 of FIGS. 9-13 in that the distal portion is enlarged and includes a curved portion at a distal end of the adapter 600. The adapter 600 is configured for use in impacting a hip bone. The curved portion of the adapter 600 may help a surgical implement releasably coupled to the distal end of the adapter 600 access the hip bone.

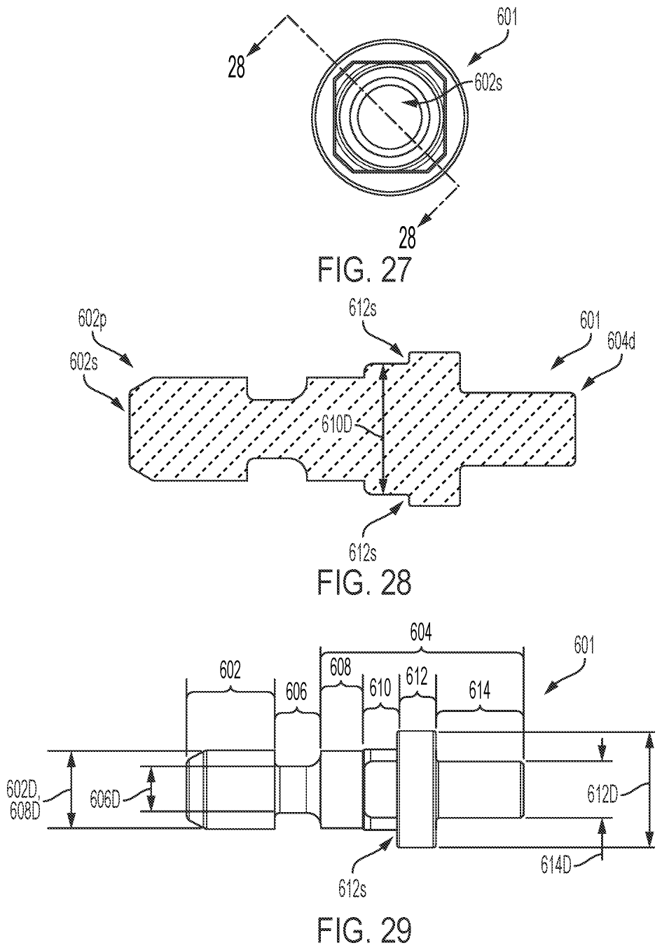

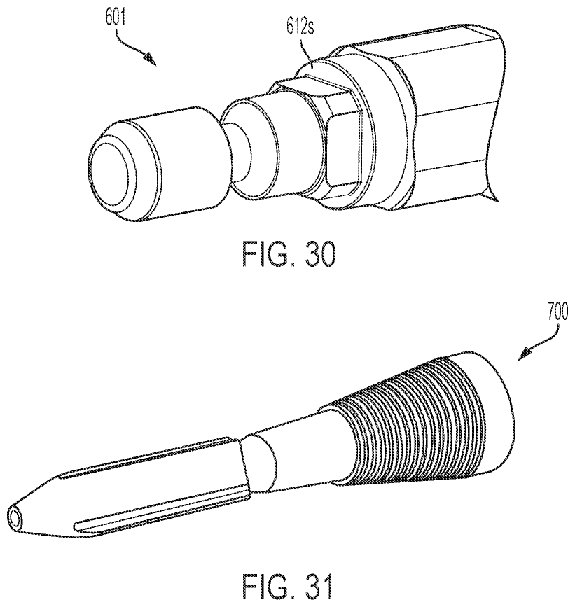

FIGS. 26-30 illustrate another embodiment of an adapter 601 configured to be releasably attached to a surgical impacting tool handpiece, such as the surgical impacting tool handpiece 100 of FIGS. 1-3, the surgical impacting tool handpiece 200 of FIGS. 4-8, or another surgical impacting tool handpiece. The adapter 601 of FIGS. 26-30 is configured and used similar to that discussed above with respect to the adapter 400 of FIGS. 9-13, e.g., includes a proximal portion 602, a distal portion 604, and an intermediate portion 606 located between the proximal and distal portions 602, 606 (see FIG. 29). However, the distal portion 606 of the adapter 601 differs from the distal portion 404 of the adapter 400 of FIGS. 9-13, and a forward or distally directed impact force is configured to be provided via an impact on a surface of the distal portion 606 instead of via an impact on a proximal-facing surface 602s the proximal portion 602.

The adapter's distal portion 606 includes first and second portions 608, 610 similar to the first and second portions 408, 410 of the adapter's distal portion 404. However, third and fourth portions 612, 614 of the adapter 401 differ from the third and fourth portions 412, 414 of the adapter's distal portion 404.

A diameter 608D of the first portion 608 of the distal portion 604 is substantially equal to a diameter 602D of the proximal portion 602, as shown in FIG. 29. A diameter 610D of the reduced diameter portion 610 is less than the diameters 608D of the proximal and first portions 602, 608. A diameter 612D of the third portion 612 is greater than the diameter 610D of the second portion and greater than a diameter 614D of the fourth portion 614. The third portion 612 therefore has a diameter 612D greater than the diameters 610D, 614D of its immediately proximal and distal portions 610, 614 and thus defines an enlarged diameter portion of the distal portion 604. The enlarged diameter of the third portion 612 provides for a forward-impact surface. In particular, the proximal-facing surface 612s of the third portion 612 defines the surface on which an element (e.g., an anvil or other element) of a surgical impacting tool handpiece can be configured to impact to provide a forward or distally directed impact force. In this illustrated embodiment the diameter 412D of the third portion 412 is about 20% greater than the maximum diameter of the adapter 400 proximal to the third portion 412, but another ratio of these diameters is possible.

The proximal portion 602 in this illustrated embodiment is chamfered at its proximal end 602p, similar to the chamfering of the proximal portion 402 of the adapter 400 of FIGS. 9-13 Additionally, in this illustrated embodiment, the distal portion 604 is chamfered at its distal end 604d. The distal portion 604 therefore has a smaller diameter at the proximal end 604d thereof than in a remainder of the distal portion's fourth portion 614 that includes the chamfering.

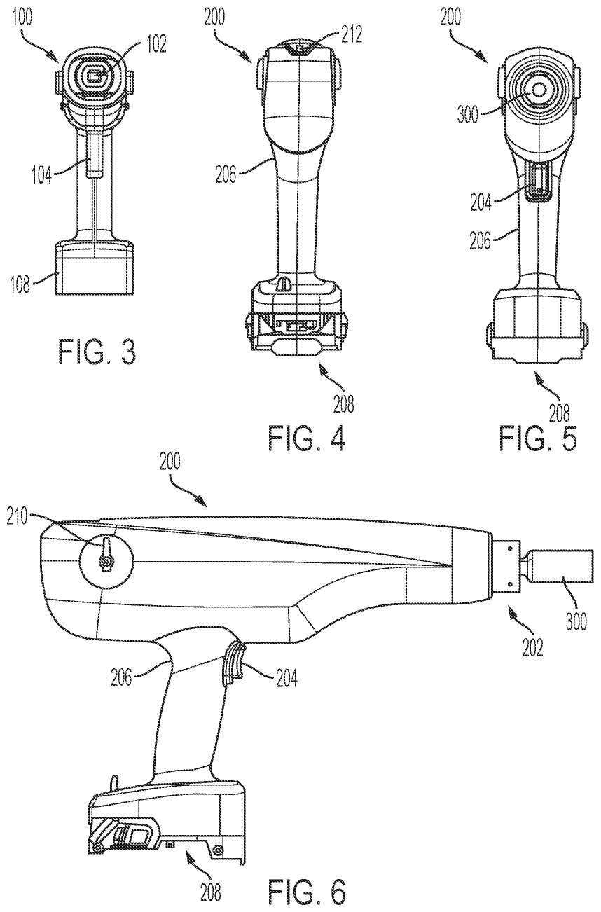

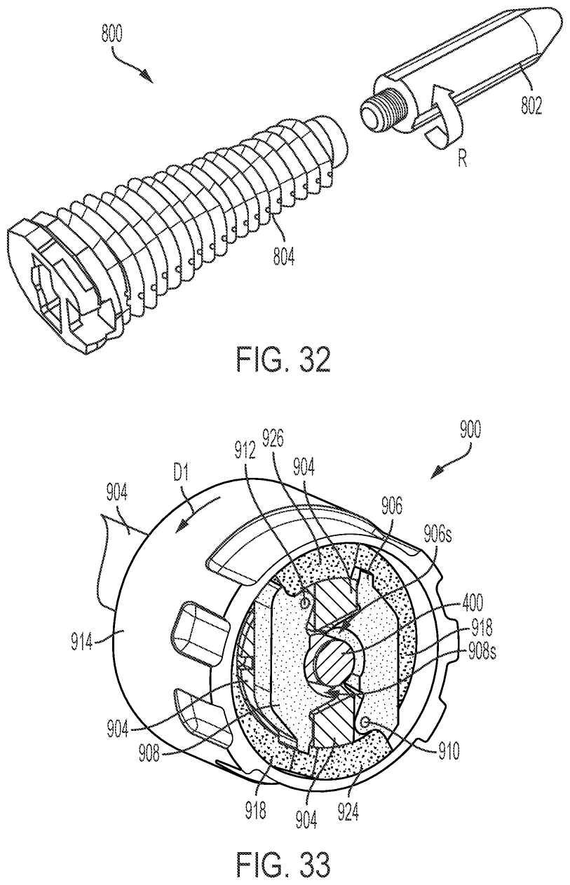

As mentioned above, various surgical implements such as chisels and broaches can be configured to releasably attach to an adapter, such as the adapter 300 of FIGS. 3 and 5-8, the adapter 400 of FIGS. 9-13, the adapter 500 of FIGS. 15-19, the adapter 600 of FIGS. 20-25, or another adapter. FIG. 31 illustrates one embodiment of a surgical implement 700 configured to releasably attach to the adapter. The surgical implement 700 in this illustrated embodiment is a tibial broach configured for impacting a tibia. FIG. 32 illustrates another embodiment of a surgical implement 800 configured to releasably attach to the adapter. The surgical implement 800 in this illustrated embodiment is a femoral broach configured for impacting a femur. The surgical implement 800 in this illustrated embodiment includes a forward or distal portion 802 and a rearward or proximal portion 804 that is configured to releasably attach to the forward portion 802 by rotating the forward portion 802 into the rearward portion 804 as shown by arrow R.

As mentioned above, an adapter can be configured to releasably attach to a surgical impacting tool handpiece via a locking assembly of the surgical impacting tool handpiece. The locking assembly can have a variety of configurations. FIGS. 33-36 illustrate one embodiment of a locking assembly 900 of a surgical impacting tool handpiece, such as the surgical impacting tool handpiece 100 of FIGS. 1-3, the surgical impacting tool handpiece 200 of FIGS. 4-8, or another surgical impacting tool handpiece. The locking assembly 900 is configured to releasably attach to an adapter. The adapter 400 of FIGS. 9-13 is shown in FIGS. 33-36, but the locking assembly 900 can be similarly used with other adapters.

The locking assembly 900 is configured to move between a locked configuration, in which the locking assembly 900 is releasably attached to an adapter, and an unlocked configuration, in which the locking assembly 900 is not releasably attached to an adapter. FIGS. 33, 34, and 36 show the locking assembly 900 in the locked configuration releasably attached to the adapter 400, and FIG. 35 shows the locking assembly 900 in the unlocked configuration.

The locking assembly 900 is biased to the locked configuration. The locking assembly 900 is configured to move to the locked configuration in response to the locking assembly's engagement with the adapter 400. The engagement of the adapter 400 with the locking assembly 900 includes the adapter 400 being moved longitudinally, or translationally, into the locking assembly 900. The adapter 400 is thus configured to move in one way, e.g., translationally and not rotationally, to attach to the locking assembly 900. The disengagement of the adapter 400 from the locking assembly 900 includes the locking assembly 900 being rotated relative to the adapter 400 and then the adapter 400 being moved longitudinally, or translationally, relative to the locking assembly 900. The adapter 400 is thus configured to move in the same one way to detach from the locking assembly 900. Rotational motion being needed to allow the adapter 400 to be detached from the locking assembly 900 (and thus from the surgical impacting tool handpiece that includes the locking assembly 900) may help prevent the adapter 400 (and a surgical implement coupled therewith) from detaching from the surgical impacting tool handpiece during impacting since the surgical impacting tool handpiece provides a longitudinally directed force for impacting that will not urge rotational movement of the locking assembly 900. The locking assembly 900 needing to rotate relative to the adapter 400 before the adapter 400 is moved translationally relative to the locking assembly 900 (and thus relative to the surgical impacting tool handpiece that includes the locking assembly 900) to be detached from the locking assembly 900 (and thus from the surgical impacting tool handpiece that includes the locking assembly 900) may help prevent a process of removing the adapter 400 (and a surgical implement attached thereto) from the locking assembly 900 (and thus from the surgical impacting tool handpiece that includes the locking assembly 900) from starting until a user intentionally rotates the locking assembly 900 since the surgical impacting tool handpiece's longitudinally directed force for impacting that will not urge rotational movement of the locking assembly 900.

The locking assembly 900 includes a cavity 902 configured to seat a rearward portion of an adapter therein. The cavity 902 is located at a forward or distal end 900f of the locking assembly 900 and thus at a forward or distal end of the surgical impacting tool handpiece that includes the locking assembly 900. The cavity 902 is formed in a base 904 of the locking assembly 900. A forward or distal portion 904f of the base 904 has the cavity 902 formed therein such that the cavity 902 is accessible at the forward end 900f of the locking assembly 900. The base 904 can be an anvil of the surgical impacting tool handpiece.

A rearward or proximal end (not shown) of the base 904 is configured to be operably coupled to a drive mechanism of the surgical impacting tool handpiece that includes the locking assembly 900 to allow the drive mechanism to provide a longitudinally directed force to the base 904 to drive impacting of a surgical implement coupled to the adapter 400 that is releasably attached to the locking mechanism 900. The drive mechanism can have a variety of configurations. Various embodiments of drive mechanisms are further described, for example, in previously mentioned U.S. Pat. Pub. No. 2013/0161050 entitled “Electric Motor Driven Tool For Orthopedic Impacting” published Jun. 27, 2013, U.S. Pat. No. 10,912,597 entitled “Orthopedic Adapter For An Electric Impacting Tool” issued Feb. 9, 2021, U.S. Pat. No. 11,083,512 entitled “Orthopedic Impacting Device Delivering A Controlled, Repeatable Impact” issued Aug. 10, 2021, U.S. Pat. Pub. No. 11,134,962 entitled “Orthopedic Impacting Device Having A Launched Mass Delivering A Controlled, Repeatable & Reversible Impacting Force” issued Oct. 5, 2021, U.S. Pat. No. 8,393,409 entitled “Electric Motor Driven Tool For Orthopedic Impacting” issued Mar. 12, 2013, U.S. Pat. No. 8,936,105 entitled “Electric Motor Driven Tool For Orthopedic Impacting” issued Jan. 20, 2015, and U.S. Pat. No. 8,695,726 entitled “Electric Motor Driven Tool For Orthopedic Impacting” issued Apr. 15, 2014.

The cavity 902 has a geometry corresponding to the geometry of the adapter's insertion portion 400i. A proximal or rearward portion 902p of the cavity 902 has a substantially cylindrical shape and a substantially circular cross-sectional shape corresponding to the substantially cylindrical shape and substantially circular cross-sectional shape of the proximal portion 402 of the adapter 400 that is part of the insertion portion 400i. An intermediate portion 902i of the cavity 902, which is distal to the proximal portion 902p of the cavity 902, has a substantially cylindrical shape and a substantially circular cross-sectional shape corresponding to the substantially cylindrical shape and substantially circular cross-sectional shape of the first portion 408 of the distal portion 404 of the adapter 400 that is part of the insertion portion 400i. A distal or forward portion 902d of the cavity 902, which is distal to the intermediate portion 902i of the cavity 902, has a substantially cube shape and a substantially square cross-sectional shape corresponding to the substantially cube shape and substantially circular square shape of the rearward portion of the second portion 410 of the distal portion 404 of the adapter 400 that is part of the insertion portion 400i.

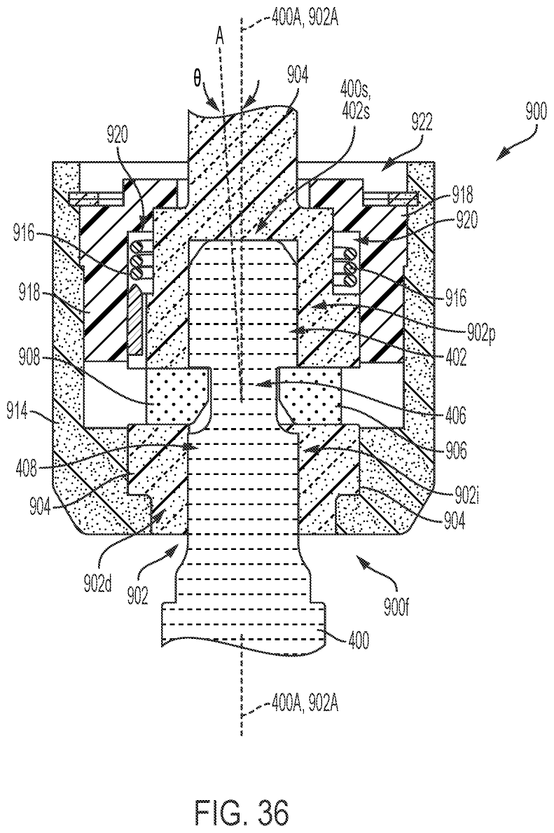

A size of the cavity 902 is just large enough to accommodate the insertion portion 400i of the adapter 400 therein, as shown in FIG. 36. With the insertion portion 400i having a size within manufacturing tolerances, and the cavity 902 having a size within manufacturing tolerances, the relative sizes and corresponding substantially cylindrical shapes of the adapter 400 and the cavity 902 allow for reduced angular play of the adapter 400 with respect to the surgical impacting tool handpiece that includes the locking assembly. FIG. 36 shows an example offset axis A that is transverse to a longitudinal axis 902A defined by the cavity 902. The offset axis A represents a longitudinal axis of the adapter 400 were the adapter 400 subject to angular play at an angle 0 while locked within the locking assembly 900. Non-cylindrical adapters and locking assembly cavities tend to result in more angular play, e.g., because of planar surfaces and/or 90° angles of components. The insertion portion 400i of the adapter 400 includes planar surfaces at the four sides 416, as discussed above, but the planar surfaces 416 are located at the forward end 900f of the cavity 902 and provide substantially zero contribution to angular play.

The locking assembly 900 is configured to seat the adapter 400 in the cavity 902 at each of a plurality of predetermined angular orientations relative to the locking assembly 900. A surgical implement coupled to the locking assembly 900 via the adapter 400, and thus also operatively coupled to the surgical impacting tool handpiece that includes the locking assembly 900, can thus be attached to the surgical impacting tool handpiece at a plurality of predetermined angular orientations relative to the surgical impacting tool handpiece. In this illustrated embodiment, the locking assembly 900 is configured to seat the adapter 400 in the cavity 902 at four predetermined angular orientations relative to the locking assembly 900 as defined by the sides 416 of the adapter 400 discussed above. Providing four predetermined angular orientations may simplify manufacturing of the base 904 that includes the cavity 902 and/or may help ensure that whether the surgical impacting tool handpiece that includes the locking assembly 900 is being held by a left hand or a right hand, and whether or not the surgical impacting tool handpiece is being held upright during impacting, the surgical implement operably coupled to the surgical impacting tool handpiece can be at a convenient angle for impacting bone. The square cube shape of the cavity 902 in the distal portion 902d thereof allows for the four predetermined angular orientations each about ninety degrees apart from one another. Other four-sided shapes, e.g., rectangle, of the cavity 902 similarly allow for four predetermined angular orientations. Other cavity shapes not having a different plural number sides will define another plural number of predetermined angular orientations. For example, a five-sided cavity shape defines five predetermined angular orientations for a corresponding five-sided adapter shape. For another example, a three-sided cavity shape defines three predetermined angular orientations for a corresponding three-sided adapter shape. For yet another example, an eight-sided cavity shape defines eight predetermined angular orientations for a corresponding eight-sided adapter shape.

The locking assembly 900 includes a first pawl 906 and a second pawl 908 that are each configured to rotate relative to the base 904 of the locking assembly 900 to lock the adapter 400 to the surgical impacting tool handpiece that includes the locking assembly 900. The first pawl 906 is attached to the base 904 at a first pivot point 910, e.g., using a pivot pin or other mechanism, about which the first pawl 906 is configured to rotate relative to the base 904. The second pawl 908 is attached to the base 904 at a second pivot point 912, e.g., using a pivot pin or other mechanism, about which the second pawl 908 is configured to rotate relative to the base 904. The first and second pivot points 910, 912 are on opposed sides of the base 904. The first and second pawls 906, 908 are configured to simultaneously pivot relative to the base 904 at their respective pivot points 910, 912.

The first and second pawls 906, 908 are positioned relative to the cavity 902 formed in the base 904 to allow the first and second pawls 906, 908 to engage the adapter 400 inserted into the cavity 902. The first and second pawls 906, 908 are opposed to one another on opposite sides of the cavity 902. The first pawl 906 defines a first longitudinal axis 906A that is substantially perpendicular to the longitudinal axis 902A defined by the cavity 902 with the locking assembly 900 in the locked configuration. As shown in FIG. 36, the longitudinal axis 902A defined by the cavity 902 is coaxial with the longitudinal axis 400A of the adapter 400 such that the first pawl's first longitudinal axis 906A is also substantially perpendicular to the adapter's longitudinal axis 400A. The second pawl 908 defines a second longitudinal axis 908A that is substantially perpendicular to the longitudinal axis 902A defined by the cavity 902, and thus also the adapter's longitudinal axis 400A, with the locking assembly 900 in the locked configuration. The locking assembly 900 moving between the unlocked configuration and the locked configuration, and with the locking assembly 900 in the unlocked configuration, the first and second longitudinal axes 906A, 908A are not substantially perpendicular to the longitudinal axis 902A defined by the cavity 902 or the longitudinal axis 400A defined by the adapter 400. The first and second longitudinal axes 906A, 908A are substantially parallel to one another with the locking assembly 900 in the unlocked configuration, with the locking assembly 900 in the locked configuration, and during movement of the locking assembly 900 between the locked and unlocked configurations. A person skilled in the art will appreciate that axes may not be precisely perpendicular or precisely parallel but nevertheless be considered to be substantially perpendicular or substantially parallel (for example, be within a +/−0.5% angular range) due to any number of factors, such as manufacturing tolerances and sensitivity of measurement equipment.

The locking assembly 900 includes a housing 914 that is operatively coupled to the first pawl 906 and the second pawl 908. The locking assembly 900 also includes a biasing element 916 (see FIG. 36) configured to bias the housing 914 in a first direction D1 (see FIG. 33), which is counterclockwise in this illustrated embodiment. The biasing element 916 biases the housing 914 to the locked configuration of the locking assembly 900. The locking assembly 900 includes a support 918 that seats the first biasing element 916 therein, as shown in FIG. 36. The support 918 is a tubular member having an inner passageway 920 extending therethrough. The biasing element 916 is disposed in the inner passageway 920. The support 918 is in fixed relation to the housing 914. The support 918 can be integrally formed with the housing 914 or can be a separate element that is fixedly attached to the housing 914.

The housing 914 is a tubular member having an inner passageway 922 extending therethrough. The cavity 902 formed in the base 904, the forward portion 904f of the base 904, the first and second pawls 906, 908, and the biasing element 916 are located in the inner passageway 922. The base 904 extends rearwardly from the housing 914 with the rearward portion of the base 904 being located outside and rearward of the housing 914.

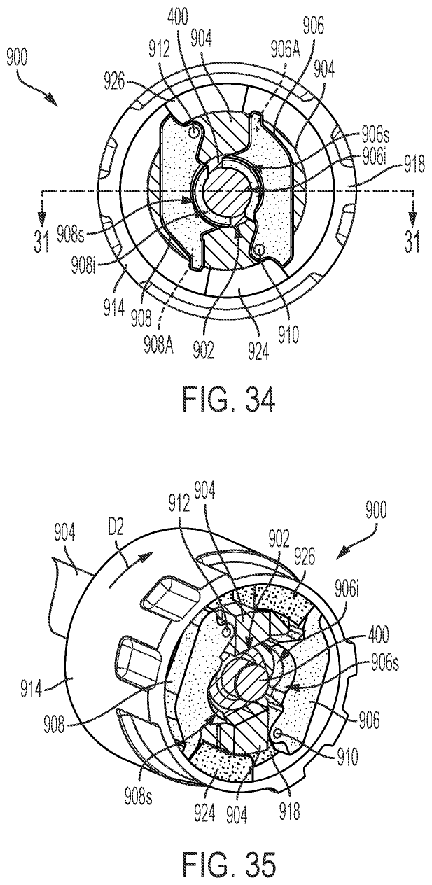

The locking assembly 900, e.g., the support 918 thereof, includes first and second bosses 924, 926 that each extend radially inward. The first and second bosses 924, 926 are configured to operatively engage the first and second pawls 906, 908 to cause pivoting of the first and second pawls 906, 908 at their respective pivot points 910, 912, as discussed further below. As shown in FIGS. 33 and 34, with the locking assembly 900 in the locked configuration, a first side surface of the first boss 924 abuts a first cam surface of the first pawl 906, and a first side surface of the second boss 926 abuts a first cam surface of the second pawl 908. The housing 914 being biased in the first direction D1 by the first biasing element 916 urges the first side surface of the first boss 924 into abutting contact with the first cam surface of the first pawl 906 and urges the first side surface of the second boss 926 into abutting contact with the first cam surface of the second pawl 908.

The locking assembly 900 is configured to automatically move to the locked configuration in response to the locking assembly's engagement with the adapter 400. In an exemplary embodiment, the adapter 400 being moved into the cavity 902 along the longitudinal axis 902A defined by the cavity 902, and thus also along the longitudinal axis 400A defined by the adapter 400, is configured to cause the locking assembly 900 to automatically move to the locked configuration. The adapter 400 can be moved into the cavity 902 by moving the adapter 400 in a rearward direction relative to the locking assembly 900, by moving the locking assembly 900 forward relative to the adapter 400, or by both moving the adapter 400 in a rearward direction relative to the locking assembly 900 and moving the locking assembly 900 forward relative to the adapter 400.

As shown in FIG. 36, the first and second pawls 906, 908 are located at an intermediate position along an axial length of the cavity 902. Thus, in moving into the cavity 902 and before being seated and locked therein, the adapter 400 encounters the first and second pawls 906, 908. First and second inside surfaces 906i, 908i of the first and second pawls 906, 908 face radially inward and face one another. The inside surfaces 906i, 908i are planar, as shown in FIGS. 35 and 36. A gap is defined between the pawl inside surfaces 906i, 908i. With the locking assembly 900 in the locked configuration, a minimum width of the gap is less than a width of the cavity 902 rearward of the pawls 906, 908 and is less than a width of the rearward portion of the adapter 400 that is being inserted into the cavity 902 and will move proximally past the pawls 906, 908, e.g., a portion of the adapter 400 including the proximal portion 402.

The first and second pawls 906, 908 also have first and second distal-facing side surfaces 906s, 908s. The distal-facing side surfaces 906s, 908s are sloped radially outward, as shown in FIGS. 33 and 34.

The adapter 400 being moved longitudinally into the cavity 902 causes the adapter 400 to engage the first and second distal-facing side surfaces 906s, 908s and then the first and second inside surfaces 906i, 908i. Because the adapter 400 in its proximal portion 402 is wider than the gap, and because the first and second pawls 906, 908 are movably attached to the base 904 having the cavity 902 formed therein, the adapter 400 can move rearwardly through the gap by slidingly engaging the pawl side surfaces 906s, 908s, which overcomes the bias of the biasing element 916 and permits the first and second pawls 906, 908 to pivot at their respective pivot points 910, 912, thereby widening the gap. The sloping of the distal-facing side surfaces 906s, 908s is configured to facilitate entry of the adapter 400 into the gap and thereby to help the adapter 400 widen the gap. The adapter 400 can thus be moved longitudinally into the cavity 902 until the rearward-facing surface 400s of the adapter 400 (which is also the rearward-facing surface 402s of the adapter's proximal portion 402) abuts a forward-facing surface of the base 904 that defines a rearward end of the cavity 902.

As discussed above, the reduced diameter portion 406 of the adapter 400 has a smaller diameter 406D than the diameter 402D of the adapter's proximal portion 402 that is rearward of the reduced diameter portion 406 and than the diameter 408D of the adapter's first portion 408 forward of the adapter's reduced diameter portion 406. The adapter's proximal portion 402 is moved rearwardly past the first and second pawls 906, 908. When the reduced diameter portion 406 of the adapter 400 becomes axially aligned with the first and second pawls 906, 908, the first and second pawls 906, 908 are no longer being urged outwardly by the adapter 400 and are allowed to freely pivot inwardly toward their initial, default position. The first and second pawls 906, 908 will thus become seated in the reduced diameter portion 602, as shown in FIGS. 33, 34, and 36. The first and second pawls 906, 908 being seated in the reduced diameter portion 406 of the adapter 400 prevents the adapter 400 from moving longitudinally relative to the locking assembly 900 (and thus relative to the surgical impacting tool handpiece that includes the locking assembly 900).

The locking assembly 900 can include a friction member (not shown) configured to add resistance to the rotational movement of the first and second pawls 906, 908. The friction member can include an o-ring but can have other configurations. The friction member may help hold the first and second pawls 906, 908 in position when the locking assembly 900 is in its unlocked configuration and in its locked configuration. A user seating the adapter 400 in the cavity 902 may be able to feel the resistance, which may help the user know that the adapter 400 is being properly attached to the locking assembly 900 (and thus to the surgical impacting tool handpiece that includes the locking assembly 900).

As mentioned above, the locking assembly 900 is configured to move from the locked configuration to the unlocked configuration. In an exemplary embodiment, the housing 914 of the locking assembly 900 being rotated about the longitudinal axis 902A defined by the cavity 902 (and thus about the adapter's longitudinal axis 400A coaxial therewith) relative to the adapter 400 and to the base 904 is configured to cause the locking assembly 900 to move from the locked configuration to the unlocked configuration to allow the adapter 400 to then be moved longitudinally along the longitudinal axis 902A defined by the cavity 902 (and thus about the adapter's longitudinal axis 400A coaxial therewith). The adapter 400 can be moved out of the cavity 902 by moving the adapter 400 in a forward direction relative to the locking assembly 900, by moving the locking assembly 900 rearward relative to the adapter 400, or by both moving the adapter 400 in a forward direction relative to the locking assembly 900 and moving the locking assembly 900 rearward relative to the adapter 400.

With the locking assembly 900 in the locked position so as to be releasably attached to the adapter 400, the housing 914 is rotated in a second direction D2, as shown in FIG. 35, that is opposite to the first direction D1 in which the housing 914 is biased. The rotation of the housing 914 in the second direction D2 also causes the support 918 fixedly attached thereto to rotate in the second direction D2, which is clockwise in this illustrated embodiment. The rotation of the housing 914 causes the first and second bosses 924, 926 of the housing 914 to rotate. The rotation of the first and second bosses 924, 926 in the second direction D2 causes the first side surface of the first boss 924 to move out of abutting contact with the first cam surface of the first pawl 906 and causes the first side surface of the second boss 926 to move out of abutting contact with the first cam surface of the second pawl 908. Continued rotation of the first and second bosses 924, 926 in the second direction D2 causes a second side surface of the first boss 924 to abut a second side surface of the second pawl 908 and causes a second side surface of the second boss 926 to abut a second side surface of the first pawl 906. Thus, the first boss 924 moves out of contact with the first pawl 906 and into contact with the second pawl 908, and the second boss 926 moves out of contact with the second pawl 908 and into contact with the first pawl 906. The rotation of the first boss 924 in the second direction D2 pushes the second pawl 908 to cause rotation of the second pawl 908 about the second pivot point 912, and rotation of the second boss 926 in the second direction D2 pushes the first pawl 906 to cause rotation of the first pawl 906 about the first pivot point 910. The gap between the first and second pawls 906, 908 thus increases, as shown in FIG. 35. The widened gap allows for the adapter 400 to be moved longitudinally out of the cavity 902 since the diameter 402D of the adapter's proximal portion 402 rearward of the reduced diameter portion 406 can now pass through the gap.

After the adapter 400 has been removed from the cavity 902, the housing 914 can be released, thereby allowing the housing 914 to rotate in the first direction D1 under force of the first biasing element 916 to return the locking assembly 900 to its initial configuration. The housing 914 can be manually moved in the first direction D1 to help the housing's rotational movement, or the housing 914 can be allowed to move in the first direction D1 fully under force provided by the biasing element 916.

In some embodiments, the base 904 can includes a blind hole rearward of and in communication with the cavity 902. A second biasing element (not shown), e.g., a coil spring, an elastomeric material, a spring-loaded plunger, etc., can be disposed in the blind hole. The second biasing element can be configured to provide a forward biasing force and can be configured to engage the rearward-facing surface 400s of the adapter 400 (and thus the rearward-facing surface 402s of the adapter's proximal portion 402) when the locking assembly 900 is locked to the adapter 400. The second biasing element can thus be configured to urge the adapter 400 in a forward direction after the housing 914 has been rotated in the second direction D2, which may help a user remove the adapter 400 from the cavity 902 by pushing the adapter 400 partially out of the cavity 902. The locking assembly 900 can omit the blind hole if a second biasing element is not present.

In some embodiments, the base 904 can include one or more protrusions extending distally from forward-facing surface of the base 904 against which the proximal-facing surface 400s of the adapter 400 (which is also the proximal-facing surface 402s of the adapter's proximal portion 402) abuts with the adapter 400 in the locked configuration. The one or more protrusions can be configured to be seated in one or more corresponding blind bores formed in the proximal-facing surface 400s of the adapter 400 (which is also the proximal-facing surface 402s of the adapter's proximal portion 402), as discussed above. In other embodiments, the base 904 can include the one or more blind bores and the rearward-facing surface 400s of the adapter 400 (which is also the rearward-facing surface 402s of the adapter's proximal portion 402) can include the one or more protrusions.

The locking assembly 900 of FIGS. 33-36 includes rotary lock pawls 906, 908 that are configured to rotate about the longitudinal axes 902A, 400A of the locking assembly's cavity 902 and the adapter 400, respectively, to facilitate unlocking of the adapter 400 from the locking assembly 900 and thus from the surgical impacting tool handpiece that includes the locking assembly 900. In another embodiment, a locking assembly configured to releasably attach to an adapter (e.g., the adapter 400 of FIGS. 9-13, the adapter 500 of FIGS. 15-19, the adapter 600 of FIGS. 20-25, the adapter 601 of FIGS. 26-30, or another adapter) includes pivoting lock pawls that are configured to pivot away from a longitudinal axis of the locking assembly's cavity and a longitudinal axis of the adapter, which is coaxial with the cavity's longitudinal axis, to facilitate unlocking of the adapter from the locking assembly and thus from the surgical impacting tool handpiece that includes the locking assembly. Various exemplary embodiments of such locking assemblies are further described, for example, in previously mentioned U.S. patent application Ser. No. 17/319,700 entitled “Surgical Impacting Tool Interfaces” filed May 13, 2021.

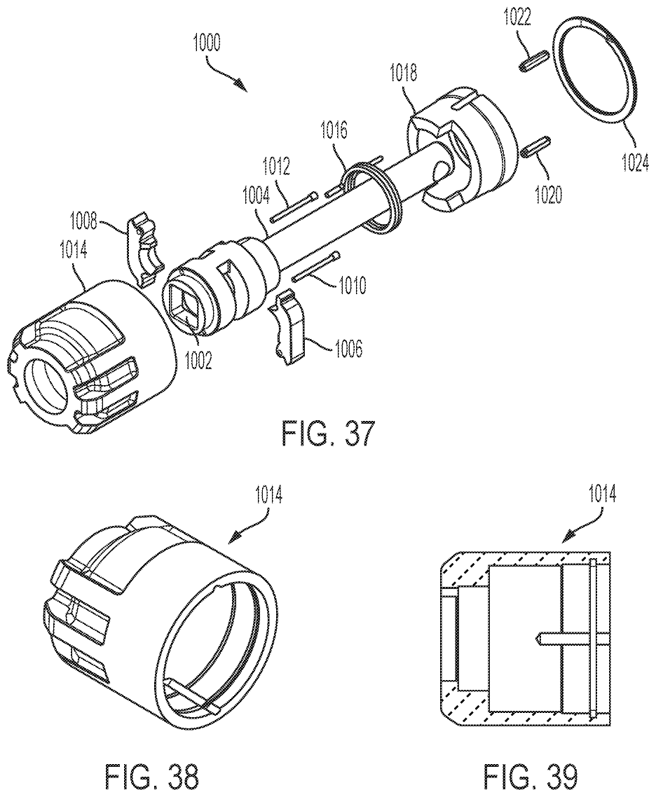

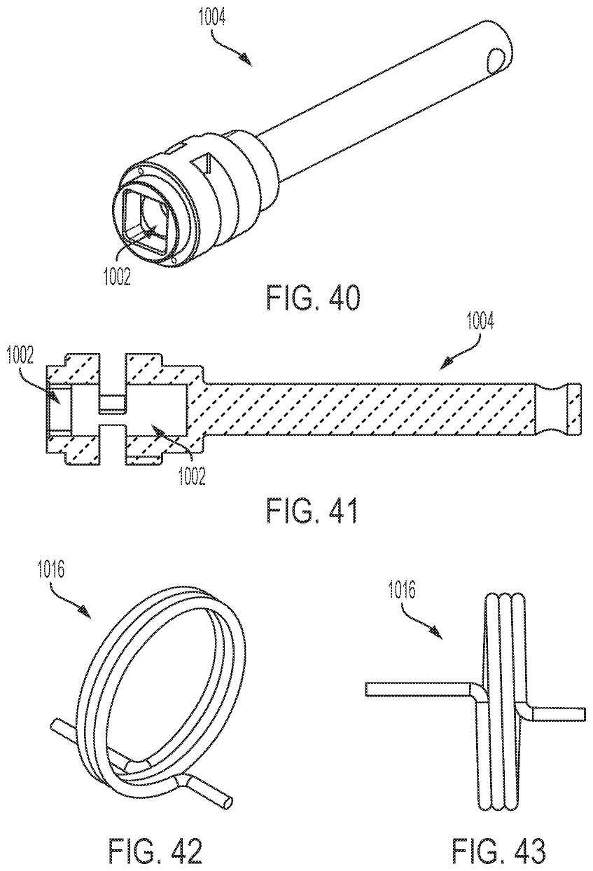

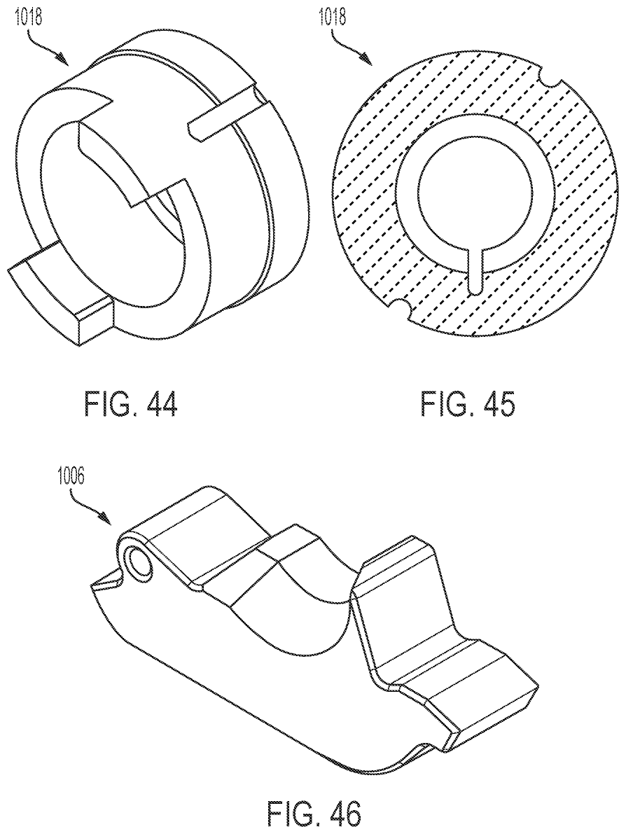

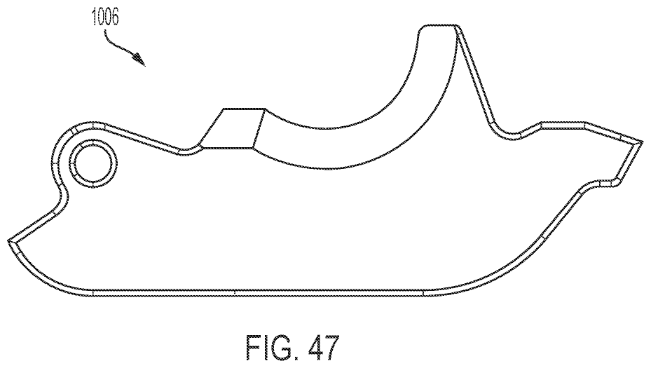

FIG. 37 illustrates another embodiment of a locking assembly 1000 of a surgical impacting tool handpiece, such as the surgical impacting tool handpiece 100 of FIGS. 1-3, the surgical impacting tool handpiece 200 of FIGS. 4-8, or another surgical impacting tool handpiece. The locking assembly 1000 is configured to releasably attach to an adapter, e.g., the adapter 400 of FIGS. 9-13, the adapter 500 of FIGS. 15-19, the adapter 600 of FIGS. 20-25, the adapter 601 of FIGS. 26-30, or another adapter. The locking assembly 1000 is configured and used similar to the locking assembly 900 of FIGS. 33-36 discussed above, e.g., includes a cavity 1002, a base 1004 (e.g., an anvil), a first pawl 1006 attached to the base 1004 at a first pivot point via a first pivot pin 1010, a second pawl 1008 attached to the base 1004 at a second pivot point via a second pivot pin 1012, a housing 1014, a biasing element 1016, and a support 1018. As shown in FIG. 37, the locking assembly 1000 also includes a first and second spring pins 1020, 1022 and a retaining ring 1024 configured to couple the biasing element 1016 to the support. FIGS. 38-47 show various elements of the locking assembly 1000 as standalone elements.

The devices disclosed herein can be designed to be disposed of after a single use, or they can be designed to be used multiple times. In either case, however, a device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

The devices described herein can be processed before use. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation or toxic gas that can penetrate the container, such as Ethylene Oxide, gamma radiation, x-rays, or high energy electrons. The radiation kills bacteria on the instrument and in the container. The sterilized instrument can then be stored in the sterile container. The sealed container keeps the instrument sterile until it is opened in a medical facility.

Sterilization can be done by any number of ways known to those skilled in the art including beta or gamma radiation, ethylene oxide, steam, and a liquid bath (e.g., cold soak).

One skilled in the art will appreciate further features and advantages of the devices, systems, and methods based on the above-described embodiments. Accordingly, this disclosure is not to be limited by what has been particularly shown and described, except as indicated by the appended claims. All publications and references cited herein are expressly incorporated herein by reference in their entirety for all purposes.

The present disclosure has been described above by way of example only within the context of the overall disclosure provided herein. It will be appreciated that modifications within the spirit and scope of the claims may be made without departing from the overall scope of the present disclosure.

1. A surgical device, comprising:
an adapter configured to have a portion thereof releasably seated in a cavity formed in a handpiece of a surgical impacting tool configured to drive impacting of bone via the adapter, wherein the adapter includes
a proximal portion having a substantially cylindrical shape, a substantially circular cross-sectional shape, and a first diameter,
a distal portion having at least four sides defining an outer perimeter of the distal portion, and the distal portion having a second diameter that is greater than the first diameter, and
a neck located between the proximal portion and the distal portion, the neck having a substantially cylindrical shape, a substantially circular cross-sectional shape, and a third diameter that is less than the first diameter and less than the second diameter.

2. The device of claim 1, wherein the proximal portion, the distal portion, and the neck are longitudinally aligned.

3. The device of claim 1, wherein:
a proximal-facing surface of the intermediate portion includes a first concave fillet connected to the neck;
a distal-facing surface of the intermediate portion includes a second concave fillet connected to the neck; and
the first concave fillet is defined by a radius greater than a radius defining the second concave fillet.

4-6. (canceled)

7. The device of claim 1, wherein:
the proximal portion is immediately proximal to the neck;
the distal portion includes a first portion having a substantially cylindrical shape and a substantially circular cross-sectional shape, the first portion being immediately distal to the neck; and
the distal portion includes a second portion that is immediately distal to the first portion and that includes the four sides.

8-9. (canceled)

10. The device of claim 1, wherein one or more of the at least four sides of the distal portion includes a marker configured to indicate an insertion level of the adapter into the cavity.

11-17. (canceled)

18. The device of claim 1, wherein a distal face of the proximal portion defines a rearward impacting surface; and
a proximal face of the proximal portion defines a forward impacting surface.

19. The device of claim 1, wherein a proximal face of the adapter is a continuous solid surface; and
the proximal face of the adapter defines a forward impacting surface.

20. The device of claim 1, wherein a proximal face of the adapter has a bore formed therein that is configured to receive therein a protrusion of the handpiece that extends into the cavity; and
the proximal face of the adapter defines a forward impacting surface.

21. The device of claim 1, wherein a distal end of the adapter is configured to releasably couple to a surgical implement configured to impact the bone.

22. The device of claim 1, wherein a distally-extending surgical implement configured to impact the bone is non-releasably coupled to the adapter.

23. A surgical device, comprising:
the adapter of claim 1; and
the handpiece of claim 1.

24-41. (canceled)

42. A surgical method, comprising:
releasably attaching the adapter of claim 1 to the handpiece by moving the adapter into the cavity of the handpiece substantially along a longitudinal axis defined by the cavity; and
impacting a surgical implement relative to bone, the surgical implement being coupled to the adapter, and a direction of the impacting being substantially along the longitudinal axis defined by the cavity.

43-50. (canceled)

51. A surgical device, comprising:
an adapter including a proximal portion configured to be releasably seated in a cavity formed in a handpiece of a surgical impacting tool, and the adapter includes a reduced diameter portion as compared to a first diameter of a first portion of the adapter proximal to the reduced diameter portion and a second diameter of a second portion of the adapter distal to the reduced diameter portion;
wherein the reduced diameter portion has a proximal fillet and a distal fillet; and
a radius of the distal fillet has a greater radius than the proximal fillet.

52. The device of claim 51, wherein the first portion has a substantially cylindrical shape and a substantially circular cross-sectional shape;
the reduced diameter portion has a substantially cylindrical shape and a substantially circular cross-sectional shape; and
the second portion of the adapter has at least four sides defining an outer perimeter of the second portion.

53. The device of claim 52, wherein the first portion has a first diameter;
the second portion has a second diameter that is greater than the first diameter; and
the reduced diameter portion has a third diameter that is less than the first diameter and less than the second diameter.

54-60. (canceled)

61. The device of claim 51, wherein the first portion, the reduced diameter portion, and the second portion are longitudinally aligned.

62. The device of claim 51, further comprising a surgical implement configured to impact bone;
wherein a distal end of the adapter is configured to releasably couple to the surgical implement.

63. The device of claim 51, wherein a distally-extending surgical implement configured to impact bone is non-releasably coupled to the adapter.

64. The device of claim 51, further comprising the handpiece;
wherein the handpiece includes first and second pawls configured to move relative to a housing of the handpiece between a first position, in which the first and second pawls are not engaged with the reduced diameter portion of the adapter, and a second position, in which the first and second pawls are not engaged with the reduced diameter portion of the adapter; and
the adapter is in a locked position relative to the handpiece with the first and second pawls in the second position.

65-67. (canceled)

68. A surgical method, comprising:
releasably attaching the adapter of claim 51 to the handpiece by moving the adapter into the cavity of the handpiece substantially along a longitudinal axis defined by the cavity; and
impacting a surgical implement relative to bone, the surgical implement being coupled to the adapter, and a direction of the impacting being substantially along the longitudinal axis defined by the cavity.

69-76. (canceled)
